(19) United States
(12) Patent Application Publication
(10) Pub. No.: US 2024/0188586 A1
(43) Pub. Date: Jun. 13, 2024

(54) MEAT-ANALOGUE COMPOSITION COMPRISING AN INTERESTERIFIED BLEND OF VEGETABLE OIL AND FULLY HYDROGENATED VEGETABLE OIL

(71) Applicant: AAK AB (publ.), Malmõ (SE)

(72) Inventors: Håkan MALMROS, Malmö (SE); Josefin LUNDIN, Malmö (SE); Charlotta TUDESJÖ, Malmö (SE); Magnus HÅKANSSON, Malmö (SE); Stefan OLOFSSON, Malmö (SE); Gabrielle GOMEZ, Malmö (SE)

(21) Appl. No.: 18/551,290

(22) PCT Filed: Mar. 22, 2022
(86) PCT No.: PCT/SE2022/050274

§ 371 Date: Sep. 19, 2023

(30) Foreign Application Priority Data
Mar. 22, 2021 (SE) 2130080-1

(51) Int. Cl. A23J 3/22; A23J 3/14

(52) U.S. Cl. CPC A23J 3/227; A23J 3/14

(57) ABSTRACT

A meat analogue composition comprises from 2% to 25% by weight of a fat composition; from 5% to 30% by weight of a non-animal protein; and from 30% to 70% by weight of water; wherein the fat composition comprises an interesterified blend of vegetable oil and fully hydrogenated vegetable oil.

### FIELD OF THE INVENTION

The invention relates to meat analogue compositions comprising a fat composition, non-animal protein and water, and the use of said meat analogue compositions in food products. In particular, the invention relates to the use of certain fat compositions in meat analogue compositions to improve various properties of the meat analogue compositions.

### BACKGROUND OF THE INVENTION

There is an increasing demand for plant-based foods due to consumer's increasing desire to eat healthy, sustainably sourced food products and to generally lower their meat intake. This has led to the development of meat-analogues; meat-free, vegetarian or vegan food products which mimic certain qualities of meat or meat-based products, such as the texture, taste and/or appearance.

Many different types of meat-analogues are available, such as those based on tofu, lentils and beans, some of which aim to mimic meat completely in terms of sizzling and browning during cooking, bleeding, colour, texture and taste. One example of such meat-analogues is plant-based burgers. Products such as plant-based sausages, meat balls, meat loaf and nuggets are also known in the art.

The typical composition of known meat-analogues is 50 to 60% water, 10 to 25% proteins (such as soy, pea, potato and wheat), 5 to 20% fat, 0 to 10% carbohydrates, as well as flavourings and colourings. Various fats have been proposed for use in meat analogue compositions. It is important that the fat is not an animal-derived fat such that the meat analogue composition is suitable for consumption by vegetarians and vegans. Accordingly, animal fats that are typically solid at room temperature are generally not used in meat analogue compositions. In order to produce a desirable meat-analogue, it is important that the final product have an appealing taste, texture and mouthfeel, and have similar taste, texture and mouthfeel to meat. Such properties are generally affected by the nature of the fat included in the meat analogue composition. The nature of the fat in meat analogue compositions also typically has an effect upon juiciness of the compositions and upon flavour release as the fats often function as carriers for fat soluble flavours. The nature of the fat is also important for the processability of the meat analogue dough such as during moulding of a meat analogue composition into burger patties. The nature of the fat is also important for providing visual similarity to meat products.

Coconut oil, palm oil, sunflower oil and rapeseed oil are examples of vegetable derived fats that have been proposed for use in meat analogue compositions. It is desirable that the fats have a relatively high melting point in order to mimic effects such as the taste, texture and mouthfeel of high melting point animal fats found in meats which are typically solid at room temperature. As a result, coconut oil and palm oil have attracted attention as they have relatively high melting points compared to other vegetable oils. Of these oils, coconut oil is typically preferred due to the negative environmental effects associated with the production of palm oil. A problem with both coconut oil and palm oil is that they are high in saturated fatty acids, which is generally considered unhealthy. The use of alternative oils lower in saturated fatty acid residues such as sunflower oil and rapeseed oil has been found to compromise desirable properties of meat analogue compositions due to the liquid nature of the oils. Properties such as juiciness are compromised and the liquid nature of the oils means that there is no structuring potential of the meat analogue composition resulting in oily meat doughs which create problems during moulding and processing of the meat analogue compositions. As a result, coconut oil remains the industry standard for the fat used in meat analogue compositions.

The inventors of the present invention have appreciated that there are various disadvantages of using coconut oil in meat analogue compositions. Firstly, as discussed above, coconut oil is high in saturated fatty acid residues which is undesirable for consumers from a health perspective due to the association of saturated fatty acid residues in fats with heart disease, undesirable cholesterol levels, and related conditions. The inventors of the present invention have also appreciated that coconut oil, despite having a relatively high melting point for a vegetable oil, has a steep melting curve. In other words at colder temperatures of less than 15° C., coconut oil is a hard brittle solid, whereas at higher temperatures of 30° C. to 35° C., the coconut oil is a liquid containing no or very little solid fat. It has been found by the inventors that the solid, hard brittle structure of coconut oil at lower temperatures means that the coconut oil is often difficult to process and sufficiently mix in with other components of the meat analogue composition during manufacture, meaning that it is sometimes desirable for the coconut oil to be melted or heated beforehand. This is undesirable in manufacturing processes due to the extra energy required to melt the coconut oil during manufacture. The inventors have also appreciated that in instances where solid, brittle particles of coconut oil are included in meat analogue compositions, said coconut oil particles have an edgy structure and appearance, which does not effectively resemble the structure of real meat where the fat particles are generally more rounded. On the other hand, where coconut oil is melted prior to inclusion in the meat analogue compositions, the resultant composition also does not effectively mimic the structure and appearance of real meat since the fat is dispersed uniformly within the composition to provide a homogenous structure and appearance which does not resemble how animal fat particles are dispersed within real meat. An additional disadvantage associated with the use of coconut oil is that its steep melting curve means that there is only a narrow temperature window in which coconut oil can be mixed into a meat analogue composition as a solid. It has also been found that having no solid fat at 30° C. to 35° C. is undesirable since this results in an overly quick release of fat/flavour from the meat analogue compositions. Many flavourants present in meat analogue compositions are fat soluble and so are released overly quickly on melting of the fat. A further disadvantage of coconut oil is that it often contains high levels of mineral oil saturated hydrocarbons (MOSH) and mineral oil aromatic hydrocarbons (MOAH).

In addition to the above, a further disadvantage of the use of coconut oil and also palm oil is that they are fats derived from plants found only in tropical regions of the world. This is disadvantageous for the manufacturers of meat-analogue compositions found outside of these regions of the world such as Europe and North America since the tropical fats need to be transported from the regions in which they are grown to Europe and North America which have the largest markets for meat analogue products. Unfortunately, the majority of naturally occurring vegetable derived fats grown locally in Europe and North America have much lower melting points than coconut oil and palm oil (such as sunflower oil etc; rapeseed oil etc.) and so are either unsuitable for inclusion in meat analogue compositions, or do not provide the advantages and functionality discussed above associated with coconut oil or palm oil. A previous approach to providing a harder, higher melting point fat from a local source for use in food products was to provide a hydrogenated vegetable oil. A locally sourced vegetable oil with a lower melting point can be hydrogenated to increase the saturated fatty acid moiety content of the fat thus increasing its melting point. Hydrogenation can be done either partly (thus leaving some unsaturated fatty acids present in the fat) or fully where 100% (or near) of the unsaturated fatty acid moieties present in the vegetable oil are converted to saturated fatty acid moieties. A disadvantage of using partially hydrogenated fats is the high content of trans unsaturated fatty acids present in them. Trans fatty acids are undesirable for health reasons as they are linked to the incidence of heart disease and higher cholesterol users in consumers, amongst other conditions. A disadvantage of fully hydrogenated fat is that the fat typically has a poor melting behaviour resulting in an undesirable and unpleasant waxy mouth feel when included in food products such as meat analogue compositions.

The inventors of the present invention have appreciated that there is a need in the art for a locally sourced non-animal derived fat that can be used in meat analogue compositions that does not have the negative health implications associated with partially hydrogenated fats, or the disadvantageous sensory properties associated with fully hydrogenated fats discussed above. The inventors have also appreciated that there is a need in the art for such fats that solve or alleviate the problems discussed above associated with the use of coconut oil in meat analogue compositions.

It has surprisingly been found by the inventors of the present invention that these problems can be solved or alleviated by the use of certain fats in meat analogue compositions instead of the use of coconut oil and other fats in such compositions.

The documents discussed below discuss the utility of certain fat compositions in certain food products. However, the use of the fat compositions in meat analogue compositions, and the possible advantages associated therewith over the state of the art are not contemplated.

EP2196094 discloses fatty products having a low amount of saturated fat mainly composed of stearic acid and a low percentage of palmitic acid, obtained through interesterification of totally hydrogenated vegetable oil with liquid vegetable oil having a very low content in saturated fatty acids. The fatty products are taught for use in manufacturing bakery products such as tarts, biscuits and bread loaves.

US2010/0015280 discloses a functional oil blend comprising less than 1.5 percent trans fatty acids, greater than 6 percent alpha-linolenic acid, less than 32 percent saturated fatty acids where less than about 16 percent of C12:0, C14:0, and C16:0 saturated fatty acids are derived from tropical oil, and a ratio of linolenic acid to alpha-linolenic acid of less than 10. The oil blends are disclosed for use in bakery shortening, spray oil, cookies and crackers.

Zero trans fats from soybean oil and fully hydrogenated soybean oil: Physico-chemical properties and food applications, Ribeiro et Al., Food Research International 42 (2009), 401 to 410 discloses an interesterified blend of fully hydrogenated soybean oil and soybean oil and suggests its use in bakery applications such as shortening and biscuit filling bases.

Similar fat blends are also available commercially and are marketed for use in bakery applications. Such fat blends include Ines 66 (an interesterified blend of sunflower oil and fully hydrogenated sunflower oil) and Rubin 20 (an interesterified blend of rapeseed oil and fully hydrogenated rapeseed oil).

### SUMMARY OF THE INVENTION

The present invention is based upon the surprising finding that certain fat compositions solve or alleviate many of the problems discussed above associated with the use of coconut oil and other fats in meat analogue compositions.

It has been found that the certain fat compositions have an improved nutritional profile relative to coconut oil due to having lower amounts of saturated fatty acid residues. Surprisingly, the inclusion of these fat compositions in meat analogue compositions in place of coconut oil has been found to not negatively affect, and in some cases improve various properties of meat analogue compositions such as various sensory properties of the compositions.

It has been found that these certain fat compositions provide improved juiciness to the meat analogue compositions when cooked or partially cooked, in comparison to meat analogue compositions comprising an equivalent amount of coconut oil or liquid oils such as sunflower oil or rapeseed oil. A further advantage of the certain fat compositions compared to coconut oil is that they can be crystallised in a more ‘plasticised form’ meaning that said compositions are more ‘deformable’ than coconut oil at typical processing temperatures meaning that said fat compositions can be incorporated and mixed into meat analogue compositions more easily. Easier processability and manufacture is thus provided.

The ability to admix the fat compositions into meat analogue compositions without melting has also been found useful by the inventors to provide heterogeneity to the surfaces of food products containing the meat analogue compositions meaning that said food products more closely mimic the visual appearance of meat, compared to meat analogue compositions where the fat is melted prior to mixing with the other components of the composition. For example, the plasticised fat structures of crystallised fat can be provided and mixed with other components of a meat analogue composition. Such plasticised fat structures have the further advantage of creating less edgy fat pieces, more resembling the appearance of intramuscular fat in real meat, in comparison to where solid coconut oil structures are included that have an edgier structure and appearance.

A further advantage of the compositions for use in the invention is that since they can have higher melting points than coconut oil, where it is desired to include solid fat particles in the meat analogue compositions, processing can be done at higher temperatures than coconut oil without the fat particles melting.

In addition to the above, the certain fat compositions are advantageous over the use of tropical fats such as coconut oil, shea oil, palm kernel oil and palm oil because such certain fat compositions are derived from vegetable fats derived from vegetable sources originating from non-tropical regions of the world such as Europe and North America. The vegetable sources originating from non-tropical regions can be grown and harvested on a commercial scale in those non-tropical regions. This is especially advantageous as Europe and North America are the biggest markets for dairy analogue products.

Additionally, the certain fat compositions are free of trans unsaturated fatty acids, meaning that said compositions are considered healthier than similar compositions comprising more significant quantities of trans fatty acids such as partially hydrogenated vegetable oils. It has also been found, advantageously, that the certain fat compositions for use in the invention do not have the undesirable “waxy” mouthfeel associated with fully hydrogenated vegetable fats and oils.

Accordingly, it has thus been found that the certain fat compositions, when used in meat analogue compositions, can provide various sensory, nutritional and functional advantages over the use of tropical fats such as coconut oil in said compositions. This is especially advantageous as the fats can be sourced from non-tropical locations which are in close proximity to major meat analogue markets. The certain fat compositions also avoid the negative health implications of partially hydrogenated fats, and the undesirable sensory properties such as waxy mouthfeel of fully hydrogenated fats.

According to a first aspect of the invention, there is provided a meat analogue composition comprising from 2% to 20% by weight of a fat composition; from 5% to 30% by weight of a non-animal protein; and from 30% to 70% by weight of water; wherein the fat composition comprises an interesterified blend of vegetable oil and fully hydrogenated vegetable oil.

In some embodiments, the fat composition comprises up to 100% by weight of the fat composition of the interesterified blend of vegetable oil and fully hydrogenated vegetable oil, such as up to 80%, 70%, 60%, 50%, 40% or 30% by weight.

In some embodiments, the fat composition comprises (a) from 5% to 95% by weight of the fat composition of the interesterified blend of vegetable oil and fully hydrogenated vegetable oil and (b) from 5% to 95% by weight of the fat composition of blending vegetable oil. For example, the fat composition may comprise (a) from 10% to 90% by weight of the fat composition of the interesterified blend of vegetable oil and fully hydrogenated vegetable oil and (b) from 10% to 90% by weight of the fat composition of blending vegetable oil. Preferably, the fat composition comprises (a) from 20% to 80% by weight of the fat composition of the interesterified blend of vegetable oil and fully hydrogenated vegetable oil and (b) from 20% to 80% by weight of the fat composition of blending vegetable oil. More preferably, the fat composition comprises (a) from 50% to 80% by weight of the fat composition of the interesterified blend of vegetable oil and fully hydrogenated vegetable oil and (b) from 20% to 50% by weight of the fat composition of blending vegetable oil. Most preferably, the fat composition comprises (a) from 60% to 80% by weight of the fat composition of the interesterified blend of vegetable oil and fully hydrogenated vegetable oil and (b) from 20% to 40% by weight of the fat composition of blending vegetable oil.

Typically, the interesterified blend is an interesterified blend of from 20% to 60% by weight of vegetable oil and from 40% to 80% by weight of fully hydrogenated vegetable oil. Preferably, the interesterified blend is an interesterified blend of from 40% to 60% by weight of vegetable oil and from 40% to 60% by weight of fully hydrogenated vegetable oil. More preferably, the interesterified blend is an interesterified blend of from 45% to 55% by weight of vegetable oil and from 45% to 55% by weight of fully hydrogenated vegetable oil.

In the context of the fat composition of the invention, the term “hardstock” is used herein to refer to the interesterified blend component of the fat composition (i.e. component (a) when a blending vegetable oil is included).

Typically, the interesterified blend is an interesterified blend of liquid vegetable oil and fully hydrogenated vegetable oil. Preferably, the interesterified blend is an interesterified blend of non-tropical vegetable oil and fully hydrogenated non-tropical vegetable oil. Non-tropical vegetable oils are those for which the sources, such as seeds or sticklings, are originated from non-tropical regions of the world such as North America, parts of Northern Asia and Europe.

In some embodiments, the interesterified blend is an interesterified blend of (i) fully hydrogenated vegetable oil and (ii) vegetable oil, each vegetable oil being selected from rapeseed oil, high oleic rapeseed oil, high erucic acid rapeseed oil, soybean oil, sunflower oil, high oleic sunflower oil, linseed oil, olive oil, corn oil, cottonseed oil, carinata oil, groundnut oil, safflower oil, high oleic safflower oil, peanut oil, rice oil, camelina oil, or any combination thereof, although it will be understood that similar vegetable oils may also be used.

Preferably, the interesterified blend is an interesterified blend of (i) fully hydrogenated vegetable oil and (ii) vegetable oil, each vegetable oil selected from rapeseed oil, high oleic rapeseed oil, high erucic acid rapeseed oil, or a combination thereof.

The term “fully hydrogenated vegetable oil” as used herein is used to refer to a vegetable oil that has undergone hydrogenation so as to convert its unsaturated fatty acid residues into saturated fatty acid residues. Suitable process conditions and methods for hydrogenating vegetable oil are known in the art. Any suitable fat hydrogenation process known in the art can be used to produce the fully hydrogenated vegetable oils of the present invention. For example, hydrogenation processes discussed in EP2196094 can be used. The term fully hydrogenated as used herein is used to distinguish the hydrogenated vegetable oils for use in producing the hardstock from partially hydrogenated vegetable oils which typically contain a significant quantity of trans fatty acid residues. In full hydrogenation, the hydrogenation process is allowed to continue to such an extent that all or substantially all of the unsaturated fatty acid residues present in the molecule are converted to saturated fatty acid residues. Accordingly, in some embodiments, the fully hydrogenated vegetable oil comprises less than 5% by weight of trans fatty acids, more preferably less than 2% by weight of trans fatty acids and most preferably less than 1% by weight of trans fatty acids, wherein said percentages of fatty acid residues refers to fatty acids bound as acyl groups in glycerides in the fully hydrogenated vegetable oil and being based on the total weight of C4 to C24 fatty acid residues bound as acyl groups present in the fully hydrogenated vegetable oil.

In some embodiments, the hardstock is derived from interesterification of a first vegetable oil and a fully hydrogenated vegetable oil that is itself derived from the first vegetable oil, although it will be understood that this is not essential. For example, in one preferred embodiment, the interesterified blend is an interesterified blend of (i) fully hydrogenated rapeseed oil and (ii) rapeseed oil.

In another preferred embodiment, the fully hydrogenated vegetable oil comprises high erucic acid rapeseed oil, or other fully hydrogenated fats with a mixed fatty acid chain length. In this embodiment, more preferably, the interesterified blend is an interesterified blend of (i) fully hydrogenated vegetable oil; (ii) fully hydrogenated high erucic acid rapeseed oil; and (iii) vegetable oil such as rapeseed oil or high oleic rapeseed oil. Most preferably, the interesterified blend is an interesterified blend of (i) from 40% to 60% by weight of vegetable oil such as rapeseed oil; (ii) from 5% to 15% by weight of fully hydrogenated high erucic acid rapeseed oil; and (iii) from 30% to 50% by weight of fully hydrogenated rapeseed oil. Other fully hydrogenated fats with a mixed fatty acid chain length include carinata oil. Without being limited by theory, it is believed that if the fully hydrogenated vegetable oil comprises a fully hydrogenated vegetable oil with a mixed fatty acid chain length, the melting behaviour of the fat is further improved for meat analogue compositions. Specifically, it is believed that the mixed fatty acid chain length alters the crystallisation pattern of the interesterified fat blend such that the melting behaviour is improved which can lead to improved sensory properties.

Typically, the interesterified blend comprises less than 55% by weight of saturated fatty acid residues, wherein said percentages of fatty acid residues refers to fatty acids bound as acyl groups in glycerides in the interesterified blend and being based on the total weight of C4 to C24 fatty acid residues bound as acyl groups present in the interesterified blend.

Typically, the interesterified blend comprises stearic acid residues in amount of from 40% to 60% and/or palmitic residues in an amount of 2.5% to 7.5%, wherein said percentages of fatty acid residues refers to fatty acids bound as acyl groups in glycerides in the interesterified blend and being based on the total weight of C4 to C24 fatty acid residues bound as acyl groups present in the interesterified blend.

In preferable embodiments, the fat composition comprises from 45% to 55% by weight of saturated fatty acid residues; from 30% to 40% of monounsaturated fatty acid residues; from 5% to 15% of polyunsaturated fatty acid residues; and/or less than 1% of trans unsaturated fatty acid residues; wherein said percentages of fatty acid residues refers to fatty acids bound as acyl groups in glycerides in the interesterified blend and being based on the total weight of C4 to C24 fatty acid residues bound as acyl groups present in the interesterified blend.

In preferable embodiments, the fat composition comprises from 40% to 50% by weight of C18:0; from 30% to 40% by weight of C18:1; from 5% to 12% of C18:2; from 1% to 6% of C18:3; and/or from 2.5% to 7.5% of C16:0; wherein said percentages of fatty acid residues refers to fatty acids bound as acyl groups in glycerides in the interesterified blend and being based on the total weight of C4 to C24 fatty acid residues bound as acyl groups present in the interesterified blend.

The interesterified blend is produced by interesterification of the fully hydrogenated vegetable oil and liquid vegetable oil. Typically, the interesterified blend has been produced by chemical interesterification, enzymatic interesterification, or a combination thereof. Any suitable interesterification process known in the art can be used to produce the interesterified blend. Suitable processes conditions for interesterification are known. For example, the interesterification process conditions discussed in EP2196094 can be used.

The fat composition also comprises blending vegetable oil component (b) which is mixed with the hardstock composition. Blending vegetable oil component (b) can be any suitable vegetable oil. Typically, blending vegetable oil component (a) is a liquid vegetable oil. Typically, blending vegetable oil component (b) is a non-tropical vegetable oil. Preferably, the blending vegetable oil of (b) comprises rapeseed oil, high oleic rapeseed oil, soybean oil, sunflower oil, high oleic sunflower oil, linseed oil, olive oil, corn oil, cottonseed oil, groundnut oil, safflower oil, high oleic safflower oil, peanut oil, rice oil, camelina oil, or any combination thereof.

The term “fat” as used herein refers to glyceride fats and oils containing fatty acid acyl groups and does not imply any particular melting point. The term “oil” is used synonymously with “fat” herein.

The term “fatty acid”, as used herein, refers to straight chain saturated or unsaturated (including mono- and poly unsaturated) carboxylic acids having 8 to 24 carbon atoms. A fatty acid having x carbon atoms and y double bonds may be denoted Cx:y. For example, palmitic acid may denoted C16:0, oleic acid may denoted C18:1. Percentages of fatty acids in compositions referred to herein include acyl groups in tri-, di- and mono-glycerides present in the glycerides and are based on the total weight of C8 to C24 fatty acids. The fatty acid profile (i.e. composition) may be determined, for example, by fatty acid methyl ester analysis (FAME) using gas chromatography according to ISO 12966-2 and ISO 12966.4.

The fat composition can be present in any suitable amount in the meat analogue composition within the limits given above. Preferably, the fat composition is present in the meat analogue composition in an amount of from 7.5% to 20% by weight of the meat analogue composition.

Preferably, the fat composition contains a substantially major portion of fat with very little water (i.e. the fat composition consists essentially of fat molecules). However, in some embodiments, the fat composition may contain water and be present in the form of an emulsion such as an oil-in-water emulsion or a water-in-oil emulsion, typically with a suitable emulsifier. In such embodiments, the weight percentage ranges provided above for the amount that the fat composition is present in the meat analogue composition refers to only fat molecules present in the fat composition, and not any water present in the composition. Similarly, the weight percentages given above for the amount of water present in the meat analogue composition refers to both water added in its own right during manufacture of the meat analogue composition, and also to any water present in other components of the meat analogue composition (such as water present in an emulsified fat composition), or water bound to any protein, as discussed in further detail below.

The meat compositions of the invention comprise one or more non-animal proteins, such as one or more proteins derived from fungi, plants, microorganisms, or a combination thereof.

Typically, the non-animal protein comprises plant protein. Preferably, the plant protein is selected from algae protein, black bean protein, canola wheat protein, chickpea protein, rapeseed protein, fava protein, lentil protein, lupin bean protein, mung bean protein, oat protein, pea protein, potato protein, rice protein, soy protein, sunflower seed protein, wheat protein, white bean protein, and protein isolates or concentrates thereof. In other embodiments, the non-animal protein comprises seitan, rice protein, mushroom protein, legume protein, tempeh, yam flour, tofu, mycoprotein, peanut flour, yuba, or a combination thereof.

More preferably, the non-animal protein comprises texturized vegetable proteins, preferably wherein the texturized vegetable proteins comprise texturized pea proteins, texturized fava proteins, texturized soy proteins, texturized wheat proteins or a combination thereof. Preferably, the texturized vegetable protein is present in the meat analogue composition in an amount of from 10% to 20% by weight of the meat analogue composition.

The non-animal protein is present in the meat analogue composition in an amount of from 5% to 30% by weight of the meat analogue composition. Preferably, the non-animal protein is present in the meat analogue composition in an amount of from 10% to 30% by weight of the meat analogue composition, and more preferably from 15% to 30% by weight of the meat analogue composition.

Plant protein is a source of protein which is obtained or derived from plants. The plant protein may be any suitable plant protein and may comprise a mixture of plant proteins and/or may include protein isolates or concentrates. Examples of suitable plant proteins include those discussed above. As discussed above, preferably, the plant protein comprises textured vegetable proteins (TVP). TVPs are extruded proteins, which may be either dry or moist (i.e. hydrated). TVP is widely available and may be made from plant sources as mentioned above, such as soy flour or concentrate. In dry form, TVP can comprise up to about 70 wt. % of protein, typically about 60 to 70 wt. % of protein, and when hydrated comprises typically about 10 to 20 wt. % of protein. Typically, when hydrated TVPs can contain up to 3 to 4 times their dry weight in water. As discussed above, the weight percentage ranges referred to above for water present in the meat analogue compositions include both water added in its own right and water present in other components of the meat analogue composition such as in textured vegetable proteins or emulsified with fat. Similarly, the weight percentage ranges given above for the amount of non-animal protein present in the meat analogue composition refer to dry weight of protein, and do not include water bound to the non-animal protein such as in textured vegetable protein.

The plant protein used in the preparation of the meat-analogue composition may be either dry (also referred to as ‘dry phase’ herein) or moist. Thus, in embodiments, the plant protein may be included in a dry mix of ingredients, which may include additional ingredients intended for inclusion in the meat-analogue composition, such as carbohydrates, fibre and/or hydrocolloids, in addition to protein. If the plant protein is dry, it may be hydrated prior to and/or during the formation of the meat-analogue composition. The term ‘dry’ used in relation to the plant protein and ‘dry phase’ used herein, is intended to mean that the phase comprising plant protein comprises less than 5 wt. % water, preferably less than 2 wt. % water, more preferably less than 1 wt. % water, even more preferably that it is substantially free from water. In other preferred embodiments, the aw of the dry phase is 0.90 or lower, more preferably below 0.80. The dry phase comprising plant protein is typically provided in a substantially dehydrated state to reduce microbial growth as far as possible so as to extend shelf life.

The meat-analogue composition comprises water, which may be added as a separate component to the composition, or derive from other components of the composition as discussed above. The amount of water is not particularly limited and, as the skilled person will appreciate, will vary depending on the intended consistency of the meat-analogue composition. Reference to ‘water’ herein is intended to include drinking water, demineralized water or distilled water, unless specifically indicated. Preferably, the water employed in connection with the present invention is demineralised or distilled water. As the skilled person will appreciate, deionized water is also a sub-class of demineralized water.

The meat analogue composition typically comprises one or more additional ingredients. Whilst these one or more additional ingredients may be preferable to include in the meat analogue compositions, it will be understood that the inclusion of the one or more additional ingredients is not essential.

The meat analogue composition preferably further comprises a stabilizer blend. Preferably, the stabilizer blend is present in the meat analogue composition in an amount of from 5% to 10% by weight of the meat analogue composition. Typically, the stabilizer blend comprises vegetable derived protein, vegetable fibre and/or a polysaccharide. Preferably, the vegetable derived protein comprises pea protein, the vegetable fibre comprises pea fibre, and/or the polysaccharide comprises methylcellulose. In highly preferred embodiments, the stabiliser blend comprises vegetable derived protein comprising pea protein, vegetable fibre comprising pea fibre, and polysaccharide comprising methylcellulose.

The meat analogue composition may comprise one or more flavouring additives. Preferably, the one or more flavouring additives are present in an amount of from 0.5% to 2% by weight of the meat analogue composition. Suitable flavouring additives known in the art may be used in the meat analogue compositions.

The meat analogue composition may comprise one or more colouring additives. Typically, the one or more colouring additives are present in an amount of from 0.5% to 5% by weight of the meat analogue composition. Suitable colouring additives known in the art may be used in the meat analogue compositions.

In some embodiments, the meat analogue composition further comprises one or more of: i) polysaccharides and/or modified polysaccharides, preferably selected from methylcellulose, hydroxypropyl methylcellulose, carboxymethyl cellulose, maltodextrin, carrageenan and salts thereof, alginic acid and salts thereof, agar, agarose, agaropectin, pectin and alginate; ii) hydrocolloids; and iii) gums, preferably selected from xanthan gum, guar gum, locust bean gum, gellan gum, gum arabic, vegetable gum, tara gum, tragacanth gum, konjac gum, fenugreek gum, and gum karaya.

Examples of other additives that may be included in the meat analogue compositions include an ionic or non-ionic emulsifier, a polyhydroxy compound, milk, liquid flavours, alcohols, humectants, honey, liquid preservatives, liquid sweeteners, liquid oxidising agents, liquid reducing agents, liquid anti-oxidants, liquid acidity regulators, liquid enzymes, milk powder, hydrolysed protein isolates (peptides), amino acids, yeast, sugar substitutes, starch, salt, spices, fibre, flavour components, colourants, thickening and gelling agents, egg powder, enzymes, gluten, vitamins, preservatives, sweeteners, oxidising agents, reducing agents, anti-oxidants, acidity regulators, or combinations thereof.

Amino acids are a preferred additive for the meat-analogue compositions of the invention, since these are known to contribute to the Maillard reaction, a form of non-enzymatic browning resulting from the chemical reaction between amino acids and sugars upon heating. This is used in flavour development of cooked foods and this reaction can be used in the meat-analogue composition to replicate the taste of meat by creating savoury meaty flavours.

In preferable embodiments, the meat analogue is suitable for consumption by vegetarians and vegans. Accordingly, in preferable embodiments, the meat analogue composition is substantially free of animal protein, and more preferably, the meat analogue composition is free of animal protein.

In preferable embodiments, the meat analogue composition is substantially free of animal-derived products, and more preferably, the meat analogue composition is free of animal-derived products.

However, in some embodiments, the meat analogue compositions may comprise animal-derived products such as animal derived proteins or fats. Accordingly, in some embodiments, the meat analogue composition further comprises one or more animal-derived products such as animal oils, marine oils, animal-derived proteins, animal-derived polysaccharides, or any combination thereof. In some embodiments, the one or more animal-derived products comprise animal milk proteins, animal milk fats, or a combination thereof. In these embodiments, the meat analogue compositions may be suitable for consumption by vegetarians on the basis that they comprise non-animal protein and proteins or fats derived from animal milk. These meat analogue compositions are suitable for consumption by vegetarians since they do not include fats or proteins derived from meat. However, it will of course be understood that such meat analogue compositions are not suitable for consumption by vegans.

In embodiments where the meat analogue compositions comprise one or more animal-derived products, the one or more animal-derived products are typically present in the meat analogue composition in an amount of from 1% to 20% by weight of the meat analogue composition.

According to a second aspect of the invention, there is provided a food product comprising a meat analogue composition of the invention. The food product may be an uncooked food product, a cooked food product, or a partially cooked food product.

Typically, the food product is a vegetarian or vegan meat substitute food product. Preferably, the vegetarian or vegan meat substitute food product is a burger, sausage, meat ball, nugget, patty, mince product, meatloaf, or other product intended to mimic conventional meat-based food products.

The properties of the meat-analogue composition or food products prepared using the composition may be measured by any suitable means. Properties of interest may include juiciness (and/or dryness), hardness, adhesiveness, springiness, cohesiveness, gumminess, chewiness and resilience. Such means include taste testers, which can provide feedback on properties of the composition or food product such as juiciness (or dryness), texture, chewiness and hardness. Typically multiple testers will be asked to mark one or more properties of the composition or food product, such as on a scale from 1 to 5. If multiple testers are asked, an average of the results can be taken to observe the general impression of the food product.

Properties of the composition or food product may also be measured using specialised equipment. For example, texture profile analysis (TPA) is a technique used to characterize textural attributes of solid and semisolid materials and may be used to determine the hardness, adhesiveness, springiness, cohesiveness, gumminess, chewiness and resilience. Gumminess is defined as the product of hardness×cohesiveness. Chewiness is defined as the product of gumminess×springiness (hardness×cohesiveness×springiness). In this technique, the test material may be compressed two times in a reciprocating motion, mimicking the chewing movement in the mouth, producing a Force versus Time (and/or distance) graph, from which the above information can be obtained. TPA and the classification of textural characteristics is described further in Bourne M. C., Food Technol., 1978, 32 (7), 62-66 and Trinh T. and Glasgow S., ‘On the texture profile analysis test’, Conference Paper, Conference: Chemeca 2012, Wellington, New Zealand, and may be performed as described therein.

The Force versus Time (and/or distance) graph typically includes two peaks in force, corresponding to the two compressions, separated by a trough. Force may be measured in gravitational force equivalent (g-force, g) or Newtons (N).

Hardness (g or N) is defined as the maximum peak force experienced during the first compression cycle.

Adhesiveness is defined as the negative force area for the first bite, i.e. the area of the graph between the two peaks in force which is at or below a force of 0 g or N. This represents the work required to overcome the attractive forces between the surface of a food and the surface of other materials with which the food comes into contact, i.e. the total force necessary to pull the compression plunger away from the sample. For materials with a high adhesiveness and low cohesiveness, when tested, part of the sample is likely to adhere to the probe on the upward stroke. Lifting of the sample from the base of the testing platform should, if possible, be avoided as the weight of the sample on the probe would become part of the adhesiveness value. In certain cases, gluing of the sample to the base of a disposable platform has been advised but is not applicable for all samples.

Springiness, also known as elasticity, is related to the height that the food recovers during the time that elapses between the end of a first compression and the start of a second compression. During the first compression, the time from the beginning of the compression at force=0 g or N to the first peak in force is measured (referred to as ‘Cycle 1 Duration’). During the second cycle, the time from the beginning of the second compression at force=0 g or N to the second peak in force is measured (referred to as ‘Cycle 2 Duration’). Springiness is calculated as the ratio of these values, i.e. ‘Cycle 2 Duration’/‘Cycle 1 Duration’.

Cohesiveness is defined as the ratio of the positive force area, i.e. the area under the curve above a force of 0 g or N, during the second compression to that during the first compression. Cohesiveness may be measured as the rate at which the material disintegrates under mechanical action. Tensile strength is a manifestation of cohesiveness. If adhesiveness is low compared with cohesiveness then the probe is likely to remain clean as the product has the ability to hold together. Cohesiveness is usually tested in terms of the secondary parameters brittleness, chewiness and gumminess.

Gumminess is defined as the product of hardness×cohesiveness and is a characteristic of semisolid foods with a low degree of hardness and a high degree of cohesiveness.

Chewiness is defined as the product of gumminess×springiness (which equals hardness×cohesiveness×springiness) and is therefore influenced by the change of any one of these parameters.

Resilience is a measurement of how the sample recovers from deformation both in terms of speed and forces derived. It is taken as the ratio of areas from the first probe reversal point, i.e. the point of maximum force, to the crossing of the x-axis, i.e. at 0 g or N, and the area produced from the first compression cycle between the start of compression and the point of maximum force. In order to obtain a meaningful value of this parameter, a relatively slow test speed should be selected that allows the sample to recover, if the sample possesses this property.

According to a third aspect of the invention, there is provided the use of a fat composition in a meat analogue composition, wherein the fat composition comprises an interesterified blend of vegetable oil and fully hydrogenated vegetable oil.

Preferably, the use further comprises using the meat analogue composition in a food product.

Preferably, the meat analogue composition, fat composition and/or food product are as described above.

The use may comprise using the fat composition to improve the nutritional profile of the meat analogue composition when compared to an analogous meat analogue composition comprising the same amount by weight of coconut oil. The term analogous meat analogue composition as used herein is used to refer to an equivalent weight of a meat analogue composition that is identical to the meat composition of the invention, with the exception of the nature of the fat present therein. The analogous meat analogue composition contains the same amount by weight of coconut oil as the meat analogue composition of the invention contains the fat composition. The nutritional profile of the meat analogue composition of the invention may be improved in comparison to coconut oil since it contains a lower total amount of saturated fatty acid residues per unit weight than coconut oil. Coconut oil contains around 90% saturated fatty acid residues. Without being limited by theory, it is believed that fats with higher saturated fatty acid contents increase the risk of heart disease, high blood pressure and associated conditions, and also have a detrimental effect upon the cholesterol levels of consumers. Accordingly, in some embodiments, the use comprises using the fat composition to improve the effect on in vivo cholesterol levels in a consumer of the meat analogue composition when compared to an analogous meat analogue composition comprising the same amount by weight of coconut oil, although it will be appreciated that other health and wellbeing benefits may also be realised by the use of the fat compositions in meat analogue compositions in place of coconut oil and similar fats.

The use may comprise using the fat composition to provide an increased resemblance to real meat of a surface or section of a cooked food product comprising the meat analogue composition when compared to an analogous food product comprising an analogous meat analogue composition comprising the same amount by weight of coconut oil. As discussed above, typically, the fat compositions are in the form of plasticized fat structures. As discussed above, a disadvantage of coconut oil is that it is a hard and brittle structure at a temperature of from 0° ° C. to 15° C., meaning that the fat particles have an edgy structure and appearance within this temperature range. In contrast, in certain meat analogue compositions of the invention, plasticized fat structures are more rounded and less edgy in shape, which more closely mimics the appearance of intramuscular fat in real meat. Accordingly, when included in meat analogue compositions of the invention, the fat compositions described herein can increase the resemblance of the meat analogue food product to real meat, compared to coconut oil, by more closely mimicking the real appearance of intramuscular fat.

As discussed above, coconut oil is often melted prior to admixture with the other components of the composition on manufacture. This results in the meat analogue composition having a more homogenous structure, which does not mimic the visual appearance of real meat. Real meat tends to have a more heterogeneous structure, with visible solid fat structures (marbling) present on the surfaces of the meat product. The fat compositions described herein can be effectively processed and mixed with the other ingredients of the meat analogue composition during manufacture without melting. This results in a substantially uniform dispersion of the fat composition in the meat analogue compositions, but with larger, solid, visible fat particles than when molten fat is included. The surfaces of food products made from the meat analogue compositions thus more closely resemble the visual appearance of real meat, and have increased heterogeneity compared to meat analogue compositions containing melted fats.

The use may comprise using the fat composition to provide improved processability to the meat analogue composition when compared to an analogous meat analogue composition comprising the same amount by weight of coconut oil, and/or an analogous meat analogue composition comprising the same amount by weight of sunflower oil. Improved processability and handleability is provided, for example, by the fat compositions having higher melting points than coconut oil, meaning that solid particles of the fat can be processed and mixed with other components of a meat analogue composition at higher temperatures without melting. Such processability is desirable, for example, when it is desired to include fat particles in the meat analogue compositions in solid form.

According to a fourth aspect of the invention, there is provided a process of manufacturing a meat analogue composition of the invention or a food product of the invention.

Preferably, the process comprises:
(a) providing a mixture of water and non-animal protein;
(b) combining the mixture from step (a) with the fat composition and optionally one or more additional components to form the meat analogue composition; and
(c) optionally forming the meat analogue composition into food products.

Typically, the process further comprises cooking the food product to form a cooked food product or partially cooked food product.

Typically, the process further comprises blending (i) the mixture of water and non-animal protein provided in step (a); (ii) a mixture formed in step (b) by combining the mixture from step (a) with the fat composition and optionally one or more additional components; and/or (iii) blending the one or more additional components with water prior to combination of the one or more additional components with the mixture from step (a) and fat composition.

Typically, the fat composition is not melted prior to combining with the mixture from step (a) and optionally one or more additional components.

Whilst the above described steps are preferable steps for manufacturing the meat analogue compositions or food products described herein, it will be appreciated that other suitable processes may also be used to manufacture the meat analogue compositions and food products.

The meat-analogue composition of the present invention may be readily prepared by blending a fat composition as described herein with plant protein and any other components of the composition. In one embodiment, there is provided a process for preparing a meat-analogue composition, said process comprising the step of: forming the meat-analogue composition by blending a plant protein with a fat composition as described herein. Optionally, further ingredients may be present. Water may be added to the composition if required at any stage during the process. The process may further comprise the step of preparing the plant protein by providing a dry phase comprising plant protein and blending the dry phase with an amount of water, which precedes the step of forming the meat-analogue composition. This step may also include other ingredients which are in dry form, such that these dry ingredients are hydrated simultaneously with the plant protein. Additionally and/or alternatively, any other dry ingredients may be hydrated separately from the plant protein in any combination. In embodiments which include TVPs, the TVP is preferably hydrated separately from any other dry ingredients. Without being bound by theory, this is believed to limit competition between the dry components for the water and ensure satisfactory hydration for all dry components present.

Thus, disclosed herein is a process for preparing a meat-analogue composition, said process comprising the steps of: a) providing a dry phase comprising plant protein and optionally any other dry ingredients of the composition and blending the dry phase with an amount of water to form a mixture; b) forming the meat-analogue composition by blending the mixture formed in step a) with a fat composition as described herein. In embodiments, the plant protein may comprise TVPs. Preferably, dry ingredients other than the plant protein are hydrated separately from the plant protein. Examples of such dry ingredients include, but are not limited to, fibres, flavours, emulsifiers, gums, hydrocolloids, thickeners.

In embodiments, the mixture of step a) comprising the hydrated plant protein and any other mixtures comprising hydrated dry ingredients are combined prior to step b). Without being bound by theory, it is believed that the hydration of dry ingredients prior to the addition of the fat composition (for example, in step a)) results in an optimal distribution of water in the product, resulting in a more stable meat-analogue composition.

The dry phase comprising plant protein used in the above process is not particularly limited. The plant protein is as described hereinabove. The term ‘dry phase’ is intended to mean that the phase comprising plant protein comprises less than 5 wt. % water, preferably less than 2 wt. % water, more preferably less than 1 wt. % water, even more preferably that it is substantially free from water. In other preferred embodiments, the aw of the dry phase is 0.90 or lower, more preferably below 0.80. The dry phase comprising plant protein is typically provided in a substantially dehydrated state to reduce microbial growth as far as possible so as to extend shelf life.

The dry phase, which may comprise plant protein, may take any physical form before being blended with water, however typically it is in powder, granule or pelletized, strip or chunk form. The amount of water added to the dry phase is not particularly limited. Typically, an amount of water is added in order to bind the dry components into a paste or dough with which the fat composition may be readily blended. The amount of water added to the dry phase is preferably calculated such that the total amount of water in the meat-analogue composition after addition of the other components of the fat composition are within the ranges described above.

The temperature of the water added is not particularly limited, so long as it does not materially impact the intended characteristics of the components (e.g. does not lead to protein denaturation or hydrolysis). In preferred embodiments, the water is below room temperature (i.e. below 20° C.). In particularly preferred embodiments, ice water is used. This is particularly preferred when water is added to the dry phase. The term “ice water” is defined herein as having a temperature of above 0° C. and below 6° C., preferably from 0.5 to 5° C., more preferably from 1 to 4° C., more preferably from 1 to 3° C. An advantage of using ice water is that it slows microbial growth as far as possible during preparation of the meat-analogue composition and it is particularly suitable for the hydration of certain dry ingredients as methylcellulose.

The blending of the dry phase with water may be performed for any duration of time. In embodiments, blending is performed until the dry phase and water are intimately mixed and typically until a paste or dough is formed. In embodiments in which TVPs are hydrated, blending is limited to a minimum so as not to overly disturb the fibrous structures. In embodiments this may be performed for a duration of from 1 minute to 30 minutes, preferably from 1 minutes to 10 minutes, more preferably from 5 seconds to 5 minutes.

Following blending of the dry phase and water, for example in step a), the mixture may be allowed to rest prior to the addition of the fat composition, for example in step b). This may ensure full hydration of the dry phase prior to addition of the fat composition. This rest may be performed under cold storage (thereby further controlling microbial growth), which has a temperature of from 0.5 to 15 ºC, preferably from 1 to 12 ºC, more preferably from 5 to 10 ºC. This rest may be performed for a duration of from 5 minutes to 5 hours, preferably from 5 minutes to 2 hours, more preferably from 5 minutes to 30 minutes.

Preparation of the meat-analogue composition may also comprise the step of adding further ingredients to the composition. These ingredients may be added at any stage in the preparation of the meat-analogue composition. In embodiments, further ingredients are added after the addition of the fat composition, for example after step b). Preferably, dry ingredients are hydrated prior to addition to the fat composition. In embodiments, dry ingredients are hydrated with any dry plant protein, such as in step a), prior to the addition of the fat composition. Such ingredients may include one or more of carbohydrates, polysaccharides, modified polysaccharides, hydrocolloids, gums, milk, liquid flavours, alcohols, humectants, honey, liquid preservatives, liquid sweeteners, liquid oxidising agents, liquid reducing agents, liquid anti-oxidants, liquid acidity regulators, liquid enzymes, milk powder, hydrolysed protein isolates (peptides), amino acids, yeast, sugar substitutes, starch, salt, spices, fibre, flavour components, colourants, thickening and gelling agents, egg powder, enzymes, gluten, vitamins, preservatives, sweeteners, oxidising agents, reducing agents, anti-oxidants, and acidity regulators, as disclosed in more detail herein. The addition of these ingredients may be performed by blending, mixing or any suitable means.

Once the meat-analogue composition has been prepared, this may be formed into a food product. This may include the step of forming the meat-analogue composition into the desired shape. The shape and size of the resulting food product is not particularly limited. Examples of shaped food products which can be made from the meat-analogue composition according to the present invention include burgers, sausages, nuggets, meatballs and mince.

Any suitable method may be used to shape the meat-analogue composition into the desired shape. In embodiments, this may be performed by cutting, moulding, pressing, extrusion, rolling, grinding or any combination thereof. These processes may be performed using an apparatus, which may be operated manually or may be automated. In embodiments, the meat-analogue composition may be compressed for 5 minutes to 24 hours, preferably 1 hour to 12 hours, more preferably 3 hours to 8 hours. The duration and pressure of compression is determined by the desired properties of the resulting food product, such as its size and density, taking into account the properties of the meat-analogue composition, such as adhesiveness, among other factors. This may form the desired shape of the food product, or it may be further processed such as by pelletizing, grinding or cutting, for instance to replicate the attributes of ground/minced meat.

The process of preparing a meat-analogue composition may further comprise cooking or part-cooking the composition, which may have been formed into a food product. Cooking may comprise boiling, baking, frying and/or microwaving. In preferred embodiments, cooking is at sufficient temperature such that the Maillard reaction may occur (for example, above 80° C. and up to 180° C., preferably from 130° C. to 170° C.). The Maillard reaction is useful for desirable browning of the food product.

### DESCRIPTION OF THE DRAWINGS

FIG. 1 shows a photograph of a burger containing the invention before (left hand side) and after cooking (right hand side).

FIG. 2 shows photographs of a comparative burger containing coconut oil before (left hand side) and after cooking (right hand side).

FIG. 3 shows photographs of a comparative burger containing high oleic sunflower oil before (left hand side) and after cooking (right hand side).

FIG. 4 shows the results of a sensory evaluation of burgers of the invention (XP 6541 compared with reference burger based on coconut oil (CNO) and high oleic sunflower oil (HOSO).

### DETAILED DESCRIPTION OF THE INVENTION

The following examples are for illustrative purposes only, and are not intended to limit the scope of the invention in any way.

### Example 1

A hardstock was prepared by interesterification of a blend of 50% by weight rapeseed oil and 50% by weight fully hydrogenated rapeseed oil.

This hardstock was mixed with rapeseed oil in an amount of 80% by weight hardstock and 20% by weight rapeseed oil to provide a fat composition for use in the present invention.

Meat analogue compositions according to the present invention were manufactured and molded into burger food products.

### General Method for Preparation of Plant-Based Burgers

The following procedure was used for the preparation of the plant-based burgers of the examples:
1. All the steps are executed on slow speed with the possibility to open the chopper plexiglass at any point.
2. Add ice water 0.6° C. (preferably ice flakes) to the meat chopper and during agitation add the cellulose. Mix for 1 minute.
3. Scrape all the cellulose together in the choppers bowl and leave to rest for 5 minutes.
4. Add proteins, spices, salt and flavouring during agitation. Mix for 1 minute.
5. Add the fat during agitation and mix for further 1 minute.
6. Stop the meat chopper and scrape down all the dough stuck around knives and in the knives house.
7. Start agitation with the blades running backwards and slowly add the hydrated protein texture. Mix for 4 minutes.
8. Stop the meat chopper and scrape down all dough stuck around knives and in the knives house.
9. Once mixed (4 minutes later) change the blades to run forward and chop them up till desired size is reached. For burgers, 15-30 seconds.
10. Let the dough rest in a fridge (operating at a temperature from 2 to 5° C.) for at least 30 minutes.
11. Form Burgers (approximately diameter 10 cm; height 1.4 cm; weight 100 g) from this dough and store in the fridge (operating at a temperature from 2 to 5° C.) prior to cooking.
12. Fry samples on a table grill at 220° C. to a core temperature of 72° C. (approximately 5-7 min).

FIG. 1 shows a photograph of a burger of the invention when uncooked (left hand side) and after cooking (right hand side). It can be seen that the cooked burger is visually acceptable and closely resembles other cooked burgers known in the art, demonstrating that meat analogue compositions of the invention can be used to provide aesthetically acceptable cooked meat analogue products.

### Example 2

Meat analogue compositions according to the present invention were manufactured and molded into burger food products as described in Example 1. Two burgers were produced (A and B).

Two comparative burgers (A and B) of the same weight as the burger of the invention (XP 6541) were also produced. The comparative burgers contained the same amount by weight of coconut oil (CNO) and high oleic sunflower oil (HOSO), respectively, as the burger of the invention comprised the fat composition.

The burgers were produced using the method described above in Example 1.

The burgers were fried until cooked, and weighed both before and after cooking.

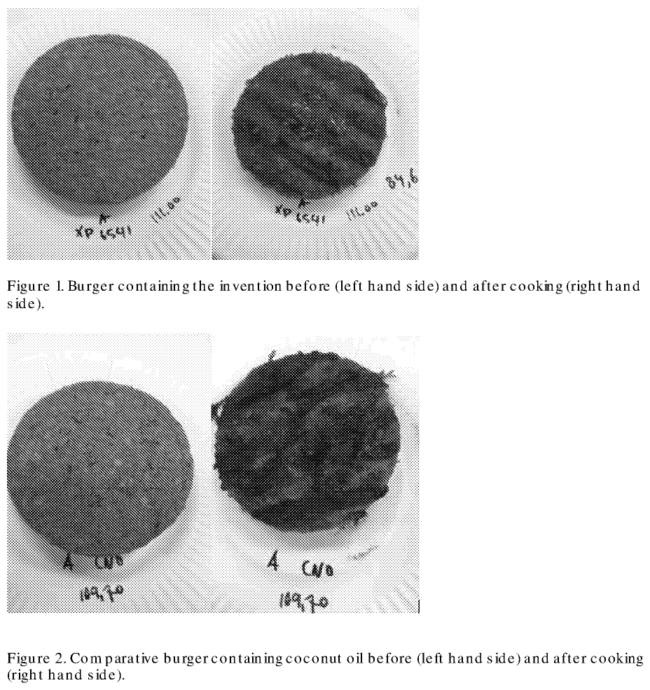

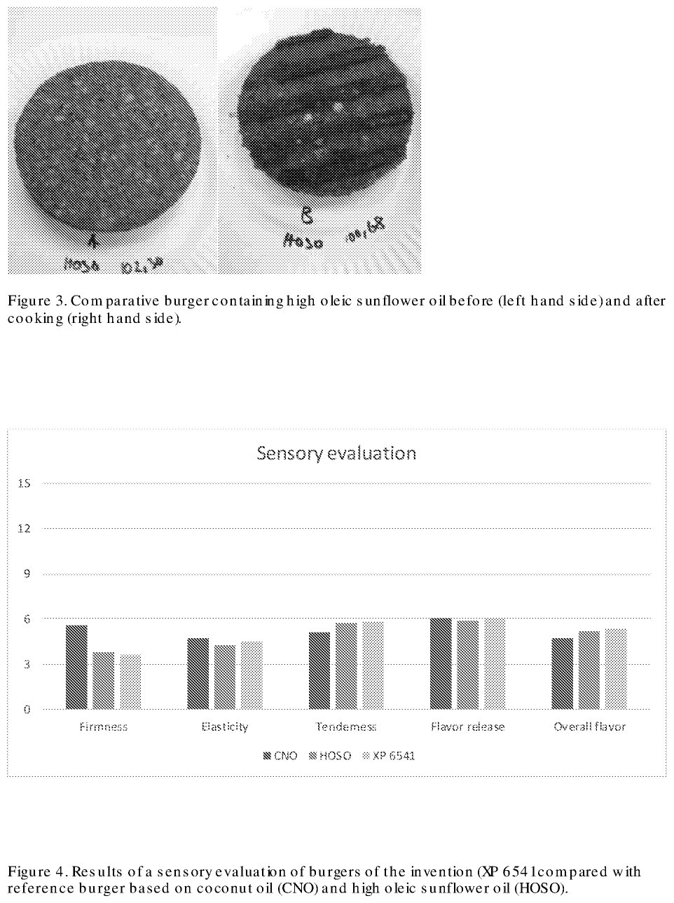

FIG. 1 shows photographs of the burger of the invention (XP 6541) before and after cooking. FIG. 2 shows photographs of the comparative burger containing coconut oil (CNO) before and after cooking. FIG. 3 shows photographs of the comparative burger containing high oleic sunflower oil (HOSO) before and after cooking.

TABLE 1

| | | Weight before | Weight after | |
|---|---|---|---|---|
| Sample | | (g) | (g) | % change |
| CNO | A | 109.70 | 82.11 | 25.2 |
| | B | 113.75 | 84.62 | 25.6 |
| | Mean | | | 25.4 |
| HOSO | A | 102.3 | 74.7 | 27.0 |
| | B | 114.72 | 73.44 | 36.0 |
| | Mean | | | 31.5 |
| XP 6541 | A | 111.0 | 84.6 | 23.8 |
| | B | 107.48 | 84.09 | 21.8 |
| | Mean | | | 22.8 |

The results in Table 1 show that the burgers of the invention demonstrate an acceptable level of weight loss upon cooking. The percentage weight loss on cooking of the burger of the invention was 22.8% whereas the percentage weight loss on cooking of the comparative burger containing coconut oil was 25.4% and the percentage weight loss on cooking of the comparative burger containing high oleic sunflower oil was 31.5%. This indicates that the invention has a lower weight loss at frying compared to the comparative burgers with coconut oil and high oleic sunflower oil, respectively.

The burgers of the invention were subjected to a sensory evaluation. The results of this sensory evaluation are depicted in FIG. 4. The results in FIG. 4 compares the burgers of the invention with two reference example burgers (containing coconut oil or high oleic sunflower oil).

It can be seen that the burgers of the invention have improved tenderness and overall flavour compared to the reference burgers. Also, the general conclusion of the panel was that no significant differences in sensation between the burgers was detected, indicating that the burger analogue compositions of the invention are an effective replacement for burger analogue compositions known in the art containing coconut oil or liquid oil.

1. A meat analogue composition comprising from 2% to 25% by weight of a fat composition; from 5% to 30% by weight of a non-animal protein; and from 30% to 70% by weight of water; wherein the fat composition comprises an interesterified blend of vegetable oil and fully hydrogenated vegetable oil.

2. A meat analogue composition according to claim 1, wherein the fat composition comprises (a) from 5% to 95% by weight of the fat composition of the interesterified blend of vegetable oil and fully hydrogenated vegetable oil and (b) from 5% to 95% by weight of the fat composition of blending vegetable oil.

3. A meat analogue composition according to claim 1, wherein the fat composition comprises (a) from 10% to 90% by weight of the fat composition of the interesterified blend of vegetable oil and fully hydrogenated vegetable oil and (b) from 10% to 90% by weight of the fat composition of blending vegetable oil.

4. A meat analogue composition according to claim 1, wherein the fat composition comprises (a) from 50% to 80% by weight of the fat composition of the interesterified blend of vegetable oil and fully hydrogenated vegetable oil and (b) from 20% to 50% by weight of the fat composition of blending vegetable oil.

5. A meat analogue composition according to claim 1, wherein the interesterified blend is an interesterified blend of from 20% to 60% by weight of vegetable oil and from 40% to 80% by weight of fully hydrogenated vegetable oil.

6. A meat analogue composition according to claim 1, wherein the interesterified blend is an interesterified blend of from 45% to 55% by weight of vegetable oil and from 45% to 55% by weight of fully hydrogenated vegetable oil.

7. A meat analogue composition according to claim 1, wherein the interesterified blend is an interesterified blend of liquid vegetable oil and fully hydrogenated vegetable oil.

8. A meat analogue composition according to claim 1, wherein the interesterified blend is an interesterified blend of non-tropical vegetable oil and fully hydrogenated non-tropical vegetable oil.

9. A meat analogue composition according to claim 1, wherein the interesterified blend is an interesterified blend of (i) fully hydrogenated vegetable oil and (ii) vegetable oil, each vegetable oil being selected from rapeseed oil, high oleic rapeseed oil, high erucic acid rapeseed oil, soybean oil, sunflower oil, high oleic sunflower oil, linseed oil, olive oil, corn oil, cottonseed oil, carinata oil, groundnut oil, safflower oil, high oleic safflower oil, peanut oil, rice oil, camelina oil, or any combination thereof.

10. A meat analogue composition according to claim 1, wherein the interesterified blend is an interesterified blend of (i) fully hydrogenated vegetable oil and (ii) vegetable oil, each vegetable oil being selected from rapeseed oil, high oleic rapeseed oil, high erucic acid rapeseed oil, or a combination thereof.

11. A meat analogue composition according to claim 1, wherein the interesterified blend is an interesterified blend of (i) fully hydrogenated rapeseed oil, fully hydrogenated high oleic rapeseed oil, or a combination thereof; and (ii) rapeseed oil, high oleic rapeseed oil, or a combination thereof.

12. A meat analogue composition according to claim 1, wherein the fully hydrogenated vegetable oil comprises high erucic acid rapeseed oil, carinata oil, or a combination thereof.

13. A meat analogue composition according to claim 12, wherein the interesterified blend is an interesterified blend of (i) fully hydrogenated rapeseed oil; (ii) fully hydrogenated high erucic acid rapeseed oil; and (iii) vegetable oil.

14. A meat analogue composition according to claim 13, wherein the interesterified blend is an interesterified blend of (i) from 40% to 60% by weight of rapeseed oil; (ii) from 5% to 15% by weight of fully hydrogenated high erucic acid rapeseed oil; and (iii) from 30% to 50% by weight of fully hydrogenated rape seed oil.

15. A meat analogue composition according to claim 1, wherein the interesterified blend comprises less than 55% by weight of saturated fatty acid residues, wherein said percentages of fatty acid residues refers to fatty acids bound as acyl groups in glycerides in the interesterified blend and being based on the total weight of C4 to C24 fatty acid residues bound as acyl groups present in the interesterified blend.

16. A meat analogue composition according to claim 1, wherein the interesterified blend comprises stearic acid residues in amount of from 40% to 60% and/or palmitic residues in an amount of 2.5% to 7.5%, wherein said percentages of fatty acid residues refers to fatty acids bound as acyl groups in glycerides in the interesterified blend and being based on the total weight of C4 to C24 fatty acid residues bound as acyl groups present in the interesterified blend.

17. A meat analogue composition according to claim 1, wherein the interesterified blend has been produced by chemical interesterification, enzymatic interesterification, or a combination thereof.

18. A meat analogue composition according to claim 1, wherein the blending vegetable oil comprises rapeseed oil, high oleic rapeseed oil, soybean oil, sunflower oil, high oleic sunflower oil, linseed oil, olive oil, corn oil, cottonseed oil, groundnut oil, safflower oil, high oleic safflower oil, peanut oil, rice oil, camelina oil, or any combination thereof.

19. A meat analogue composition according to claim 1, wherein the nonanimal protein comprises protein derived from fungi, plants, microorganisms, or a combination thereof.

20. A meat analogue composition according to claim 1, wherein the nonanimal protein comprises plant protein.

21. A meat analogue composition according to claim 1, wherein the nonanimal protein comprises seitan, rice protein, mushroom protein, legume protein, tempeh, yam flour, tofu, mycoprotein, peanut flour, yuba, or a combination thereof.

22. A meat analogue composition according to claim 1, wherein the nonanimal protein comprises texturized vegetable proteins.

23. A meat analogue composition according to claim 22, wherein the texturized vegetable protein is present in the meat analogue composition in an amount of from 10% to 20% by weight of the meat analogue composition.

24. A meat analogue composition according to claim 1, wherein the meat analogue composition comprises a stabilizer blend.

25. A meat analogue composition according to claim 24, wherein the stabilizer blend is present in the meat analogue composition in an amount of from 5% to 10% by weight of the meat analogue composition.

26. A meat analogue composition according to claim 24, wherein the stabilizer blend comprises vegetable derived protein, vegetable fibre and a polysaccharide.

27. A meat analogue composition according to claim 1, wherein the meat analogue composition comprises one or more flavouring additives.

28. A meat analogue composition according to claim 1, wherein the meat analogue composition comprises one or more colouring additives, wherein the one or more colouring additives are present in an amount of from 0.5% to 5% by weight of the meat analogue composition.

29. A meat analogue composition according to claim 1, wherein the composition further comprises one or more of: i) polysaccharides and/or modified polysaccharides; ii) hydrocolloids; and iii) gums.

30. A meat analogue composition according to claim 1, wherein the meat analogue composition further comprises an ionic or non-ionic emulsifier, a polyhydroxy compound, milk, liquid flavours, alcohols, humectants, honey, liquid preservatives, liquid sweeteners, liquid oxidising agents, liquid reducing agents, liquid anti-oxidants, liquid acidity regulators, liquid enzymes, milk powder, hydrolysed protein isolates (peptides), amino acids, yeast, sugar substitutes, starch, salt, spices, fibre, flavour components, colourants, thickening and gelling agents, egg powder, enzymes, gluten, vitamins, preservatives, sweeteners, oxidising agents, reducing agents, anti-oxidants, and acidity regulators.

31. A meat analogue composition according to claim 1, wherein the fat composition is present in the meat analogue composition in an amount of from 7.5% to 25% by weight of the meat analogue composition.

32. A meat analogue composition according to claim 1, wherein the nonanimal protein is present in the meat analogue composition in an amount of from 10% to 30% by weight of the meat analogue composition.

33. A meat analogue composition according to claim 1, wherein the meat analogue composition is substantially free of animal protein.

34. A meat analogue composition according to claim 1, wherein the meat analogue composition is substantially free of animal-derived products.

35. A meat analogue composition according to claim 1, wherein the meat analogue composition further comprises one or more animal-derived products.

36. A meat analogue composition according to claim 35, wherein the one or more animal-derived products comprise animal milk proteins, animal milk fats, or a combination thereof.

37. A meat analogue composition according to claim 35, wherein the one or more animal-derived products are present in the meat analogue composition in an amount of from 1% to 20% by weight of the meat analogue composition.

38. A food product comprising a meat analogue composition according to claim 1.

39-46. (canceled)

47. A process of manufacturing a meat analogue composition according to claim 1, wherein the process comprises:
(a) providing a mixture of water and non-animal protein;
(b) combining the mixture from step (a) with the fat composition and one or more additional components to form the meat analogue composition; and
(c) forming the meat analogue composition into food products.

48-50. (canceled)
